(19) United States
(12) Patent Application Publication
(10) Pub. No.: US 2006/0247599 A1
(43) Pub. Date: Nov. 2, 2006

(54) Garment having an outer shell that freely moves in relation to an absorbent assembly therein

(75) Inventors: Erica Leigh Mullen, Oshkosh, WI (US); Cynthia Helen Hendren, Winneconne, WI (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 11/118,619

(22) Filed: Apr. 29, 2005

(51) Int. Cl. A61F 13/15

(52) U.S. Cl. 604/393; 604/396; 604/402

(57) ABSTRACT

An absorbent garment having a garment-like outer shell and an absorbent assembly positioned therein as disclosed. In particular embodiments, the outer cover of the absorbent assembly has a relatively low coefficient of friction with the interior side of the garment shell. In one embodiment, the garment shell may be constructed such that the coefficient of friction between the garment shell and the outer cover of the absorbent assembly is less than, such as at least 15 percent less than the coefficient of friction between the exterior side of the garment shell and an adjacent material. The adjacent material may include, for instance, a standard bed sheet material. By carefully controlling the frictional properties of the materials as described above, problems associated with bunching and twisting of the garment are minimized.

### BACKGROUND OF THE INVENTION

Personal wear garments and other articles find widespread use as personal care products including, without limitation, diapers, children's toilet training pants, disposable youth pants, adult incontinence garments, sanitary napkins and the like, as well as surgical bandages and sponges. The primary purpose of such articles is to take in and retain body exudates released by a wearer to thereby prevent soiling of the wearer's or caregiver's clothing. Certain absorbent articles are suitably disposable in that they are intended to be discarded after a limited period of use, i.e., the articles are not intended to be laundered or otherwise restored for reuse.

Recently, various attempts have been made to make absorbent articles such as children's training pants and adult incontinence garments more visually appealing, such as by applying certain graphics or other features which make the pants appear more like conventional clothing, and more particularly like conventional undergarments. Training pants represent an intermediate stage for a child between using diapers and using cloth underpants. By making the training pants more closely resemble the undergarments or other clothing that an older sibling or parent wears, it is believed that children ready for toilet training will be more amenable to wearing the training pants. In another example, some children require the use of nighttime disposable absorbent pants to address bed-wetting problems. Children requiring these absorbent pants generally desire the pants they are wearing to be as discreet as possible. Wearing an absorbent garment that resembles conventional clothing can be a significant benefit for such children.

In this regard, various boxer-type or skirt-type garments have been proposed that include an absorbent assembly attached to or integral with an outer shell garment. The absorbent assembly is designed to take in or absorb body exudates. The outer shell garment, however, disguises the absorbent assembly giving the garment the appearance of conventional clothing.

In some applications, the outer shell fits loosely and drapes over the absorbent assembly. Unfortunately, the loose fitting nature of the outer shell provides the potential for twisting and bunching of the garment which can not only lead to discomfort but can also cause repositioning of the absorbent assembly. The problems associated with twisting and bunching are particularly prevalent when the garment is worn to bed. Specifically, the interaction between the garment and the bed sheets can lead to the twisting and bunching as described above. As such, a need currently exists for a boxer-type or skirt-type absorbent garment that minimizes bunching and twisting while the product is being worn.

### SUMMARY OF THE INVENTION

In general, the present disclosure is directed to a boxer-like or skirt-like absorbent garment. The absorbent garment includes an absorbent assembly surrounded by a loose fitting outer shell. The outer shell, for instance, may have two leg openings and have the appearance of a pair of boxer shorts. Alternatively, the outer shell may include only a single opening for receiving both legs and thus appear more like a skirt. The entire garment may be configured to be disposable after a single use or, alternatively, may have an outer shell that is reusable while only the absorbent assembly is disposable. In accordance with the present invention, the coefficient of friction between the loosely fitting outer shell and the exterior surface of the absorbent assembly are controlled in order to prevent twisting and bunching. In other embodiments, the outer shell is also configured to have a coefficient of friction when placed against normal bedding materials that also prevents twisting and bunching.

In one particular embodiment, for instance, the present disclosure is directed to an absorbent garment comprising a garment shell having a waist opening and at least one opposing leg opening for receiving the legs of a wearer. The garment shell includes an interior and an exterior and has a first side facing towards the interior and a second side facing towards the exterior.

An absorbent assembly is positioned on the interior of the garment shell. The absorbent assembly is attached to the garment shell in a manner that allows substantial portions of the garment shell to move freely with respect to the absorbent assembly. The absorbent assembly includes an outer cover surrounding an absorbent member. For example, in one embodiment, the absorbent assembly includes an absorbent member positioned in between an outer cover and a liquid permeable liner.

In order to prevent twisting and bunching, the outer cover of the absorbent assembly and the first side of the garment shell may have a static coefficient of friction between them of less than about 0.7, such as from about 0.3 to about 0.7, such as from about 0.3 to about 0.4 and may have a kinetic coefficient of friction between them of less than about 0.6, such as from about 0.2 to about 0.6, such as from about 0.2 to about 0.4.

As described above, in addition to selecting materials so that the outer shell has a particular static coefficient of friction with respect to the outer cover of the absorbent assembly, in some embodiments, it may also be important such that the outer shell has a particular coefficient of friction against standard bedding materials, such as standard sheet materials. In general, the coefficient of friction between the outer cover of the absorbent assembly and the first side of the garment shell should generally be less than the coefficient of friction between the second side of the garment shell and a standard sheet material. For instance, in one embodiment, the second side of the garment shell may be configured to have a static coefficient of friction of from about 0.5 to about 0.9, such as from about 0.35 to about 0.45 when tested against a standard cotton/polyester sheet material. The second side of the garment shell may also have a kinetic coefficient of friction of from about 0.4 to about 0.8, such as from about 0.4 to about 0.6 when also tested against a standard cotton/polyester sheet material.

The manner in which the absorbent assembly is positioned on the interior of the garment shell can vary and is generally not critical to the present invention. For instance, the absorbent assembly may be removably secured to the garment shell or may be permanently affixed to the garment shell. In one embodiment, for instance, the absorbent assembly includes a front end portion and a back end portion. The front end portion and the back end portion can be connected to the waist area of the garment shell. In particular, the front end portion and the back end portion can be connected to the waist area of the garment shell using any suitable thermal or adhesive bonding or may be attached using hook and loop type fasteners.

The materials that are used to form the absorbent assembly and garment shell can also vary widely depending upon the particular application. For instance, the outer cover of the absorbent assembly may comprise any suitable disposable material, such as a spunbond web, a meltblown web, a film or mixtures thereof.

The garment shell, on the other hand, may comprise any suitable material that is either disposable or reusable. For instance, when reusable, the garment shell may comprise a woven fabric. In other applications, however, the garment shell may be constructed so as to be disposed of after a single use. In this embodiment, the outer shell may comprise only a spunbond web or a laminate containing a spunbond web. The laminate may be, for instance, a spunbond/meltblown/spunbond laminate or a spunbond/meltblown laminate. In one particular embodiment, the garment shell comprises two layers of a laminate as described above bonded together. The two laminate layers may be bonded together over their entire surface area or may be point bonded together.

As described above, in many embodiments, the coefficient of friction between the absorbent assembly and the garment shell can be less than the coefficient of friction between the garment shell and a standard sheet material. For example, in one embodiment, the static coefficient of friction between the first side of the garment shell and the outer cover of the absorbent assembly may be at least about 15%, such as at least about 20% and, in one embodiment, at least about 25% less than the static coefficient of friction between the second side of the garment shell and a standard cotton/polyester sheet material. Similarly, the kinetic coefficient of friction between the first side of the garment shell and the outer cover of the absorbent assembly is also at least about 15%, such as at least about 20% and, in one embodiment, at least about 25% less than the kinetic coefficient of friction between the second side of the garment shell and a standard cotton/polyester sheet material.

Other features and aspects of the present invention will be discussed in greater detail below.

### DEFINITIONS

Within the context of this specification, each term or phrase below will include the following meaning or meanings.

“Coefficient of friction” means coefficient of friction testing conducted using a TMI slip and friction tester available from Testing Machines, Inc. of Islanda, N.Y. and according to test procedure STM 4502. Samples were conditioned at 23° C.±1° C. and 50±2% relative humidity for a minimum of four hours prior to testing. Specific test parameters were as follows:

| Delay | 3 | seconds |
|---|---|---|
| Sled | 200 grams, 6.35 × 6.35 cm | |
| Static Duration | 10,000 | ms |
| Static Speed | 15.25 | cm/min. |
| Kinetic Speed | 15.25 | cm/min |
| Kinetic Length | 15 | cm |

“Longitudinal,” and “transverse” or “lateral,” have their customary meaning, as indicated by the longitudinal and transverse axes depicted in FIG. 4. The longitudinal axis lies in the plane of the article and is generally parallel to a vertical plane that bisects a standing wearer into left and right body halves when the article is worn. The transverse or lateral axis lies in the plane of the article generally perpendicular to the longitudinal axis.

“Meltblown” refers to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface. Meltblown fibers used in the present invention are preferably substantially continuous in length.

“Non-woven” as used in reference to a material, web or fabric refers to such a material, web or fabric having a structure of individual fibers or threads that are interlaid, but not in a regular or identifiable manner as in a knitted fabric. Non-woven materials, fabrics or webs have been formed from many processes such as, for example, meltblowing processes, spunbonding processes, air laying processes, and bonded carded web processes. The basis weight of non-wovens is usually expressed in ounces of material per square yard (osy) or grams per square meter (gsm) and the fiber diameters are usually expressed in microns. (Note: to convert from osy to gsm, multiply osy by 33.91.).

“Spunbonded fibers”, or “spunbond fibers”, means small-diameter fibers that are typically formed by extruding molten thermoplastic material as filaments from a plurality of fine capillaries of a spinneret having a circular or other configuration, with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, U.S. Pat. No. 3,502,538 to Petersen, and U.S. Pat. No. 3,542,615 to Dobo et al., each of which is incorporated by reference in its entirety and in a manner consistent with the present document. Spunbond fibers are quenched and generally not tacky when they are deposited onto a collecting surface. Spunbond fibers are generally continuous and often have average diameters larger than about 7 microns, and more particularly between about 10 and 30 microns. A spunbond material, layer, or substrate comprises spunbonded (or spunbond) fibers.

“Standard cotton/polyester sheet material” means 180 thread count soft percale bed sheets containing 50% cotton and 50% polyester. Such bed sheets are available from numerous commercial sources.

“Stretch bonded” refers to an elastic member being bonded to another member while the elastic member is extended at least about 25 percent of its relaxed length. More suitably, the term “stretch bonded” refers to the situation wherein the elastic member is extended at least about 100 percent, and even more suitably at least about 300 percent, of its relaxed length when it is bonded to the other member.

“Stretch bonded laminate” refers to a composite material having at least two layers in which one layer is a gatherable layer and the other layer is an elastic layer. The layers are joined together when the elastic layer is in an extended condition so that upon relaxing the layers, the gatherable layer is gathered.

These terms may be further defined with additional language in the remaining portions of the specification.

### BRIEF DESCRIPTION OF THE DRAWINGS

FIG. 1 is a front view of an absorbent garment according to one embodiment of the present invention;

FIG. 2 is a perspective view of the absorbent garment of FIG. 1 with a side seam of the absorbent garment shown in a partially fastened, partially unfastened condition;

FIG. 3 is a plan view of an absorbent assembly of the absorbent garment of FIG. 1 with the absorbent assembly shown in an unfastened, stretched and laid flat condition, and showing the surface of the absorbent assembly that faces the wearer of the absorbent garment, and with portions cut away to show underlying features;

FIG. 3a is a plan view similar to FIG. 3, but showing the surface of the absorbent that faces away from the wearer of the absorbent garment;

FIG. 4 is a plan view similar to FIG. 3a illustrating an alternative embodiment of an absorbent assembly;

FIG. 5 is an elevated side view of one embodiment of the absorbent garment of the present invention, with a side seam of the absorbent garment shown in an unfastened condition;

FIG. 6 is a side cross-sectional view of the absorbent garment of FIG. 5;

FIG. 7 is an elevated side view of another embodiment of the absorbent garment of the present invention, with a side seam of the absorbent garment shown in an unfastened condition;

FIG. 8 is an elevated side view of another embodiment of the absorbent garment of the present invention, with a side seam of the absorbent garment shown in an unfastened condition;

FIG. 9 is an elevated side view of another embodiment of an absorbent garment incorporating an absorbent assembly similar to the one shown in FIG. 4, with a side seam of the absorbent garment shown in an unfastened condition and one pair of side panels of the absorbent assembly also shown in an unfastened condition; and

FIG. 10 is an exploded perspective view of another embodiment of the absorbent garment of the present invention.

Corresponding reference characters indicate corresponding parts throughout the drawings.

### DETAILED DESCRIPTION

It is to be understood by one of ordinary skill in the art that the present discussion is a description of exemplary embodiments only, and is not intended as limiting the broader aspects of the present invention.

In general, the present disclosure is directed to absorbent garments that are configured to readily absorb body exudates released by the wearer, but yet have the appearance of conventional clothing. In particular, the absorbent garments have a boxer-like or skirt-like outer shell that drapes over an inner absorbent assembly that is positioned next to the crotch of the wearer.

In accordance with the present invention, in order to prevent twisting and bunching of the garment and/or to prevent any other unwanted interaction between the outer shell and the interior absorbent assembly, the coefficient of friction between the outer shell and the absorbent assembly is carefully controlled within determined limits. For example, in one embodiment, the exterior surface of the absorbent assembly and the garment shell have a static coefficient of friction of less than about 0.7, such as between about 0.3 to about 0.7, such as from about 0.3 to about 0.4. The kinetic coefficient of friction between the exterior surface of the absorbent assembly and the garment shell can be less than about 0.6, such as from about 0.2 to about 0.6, such as from about 0.2 to about 0.4.

In addition to having a relatively low coefficient of friction between the garment shell and the absorbent assembly, the garment shell should also have a somewhat relatively low coefficient of friction between itself and materials that are expected to come into contact with the garment, such as bedding materials when the garments are worn at night. In order to prevent twisting and bunching of the garment, the coefficient of friction between the absorbent assembly and the garment shell should also generally be less than the coefficient of friction between the garment shell and an adjacent material, such as bed sheets. By having the coefficient of friction between the garment shell and an adjacent material be greater than the coefficient of friction between the garment shell and the absorbent assembly, the present inventors have discovered that, in this configuration, the garment shell moves freely against the absorbent assembly which is found to prevent the problems associated with bunching and twisting of the garment.

In one embodiment, for instance, the static coefficient of friction between the garment shell and a standard bed sheet may be from about 0.5 to about 0.9, such as from about 0.6 to about 0.7. The kinetic coefficient of friction between the above two materials can be from about 0.4 to about 0.8, such as from about 0.4 to about 0.6.

The sheet material that is tested against the garment shell may vary. In one embodiment, the garment shell may be tested against a standard 180 thread count cotton/polyester sheet. The sheet may contain, for instance, 50% cotton and 50% polyester. In other embodiments, however, the garment shell may be tested against 100% cotton Jersey knit sheets or 100% cotton heavyweight flannel sheets. The above three materials account for most of the bedding materials that are used by small children.

As described above, the static or kinetic coefficient of friction between the garment shell and the absorbent assembly, in one embodiment, may be less than the static and kinetic coefficient of friction between the outer shell and an adjacent material, such as a bed sheet. In various embodiments, for instance, the coefficient of friction between the garment shell and the absorbent assembly may be at least about 15%, such as at least about 20%, and, in one embodiment, at least about 25% less than the coefficient of friction between the garment shell and the adjacent material.

Various embodiments of absorbent garments made in accordance with the present invention will now be discussed in detail. In general, the absorbent garment includes an absorbent assembly surrounded by a garment shell. The absorbent assembly is attached to the garment shell in a manner that allows substantial portions of the garment shell to move freely with respect to the absorbent assembly. The manner in which the absorbent assembly is associated with the garment shell, however, is generally not critical to the present invention. Thus, the following description and associated figures merely represent optional embodiments for better describing the features and aspects of the present invention.

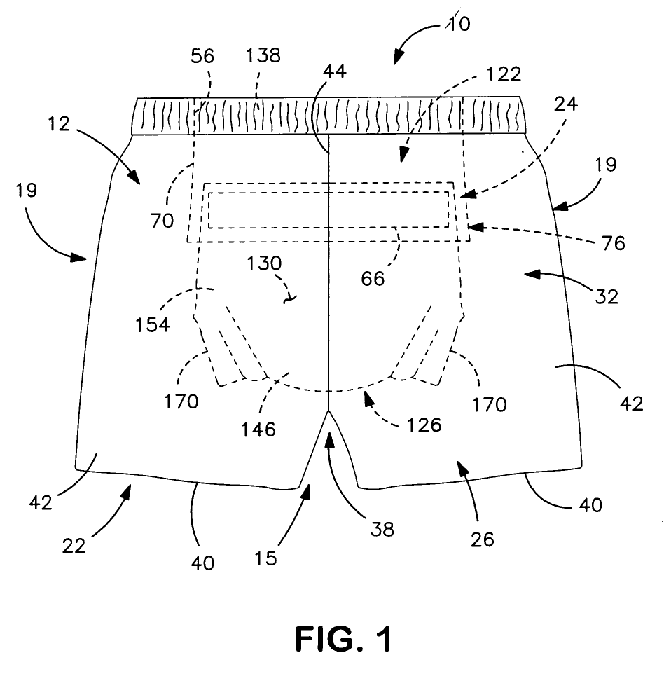

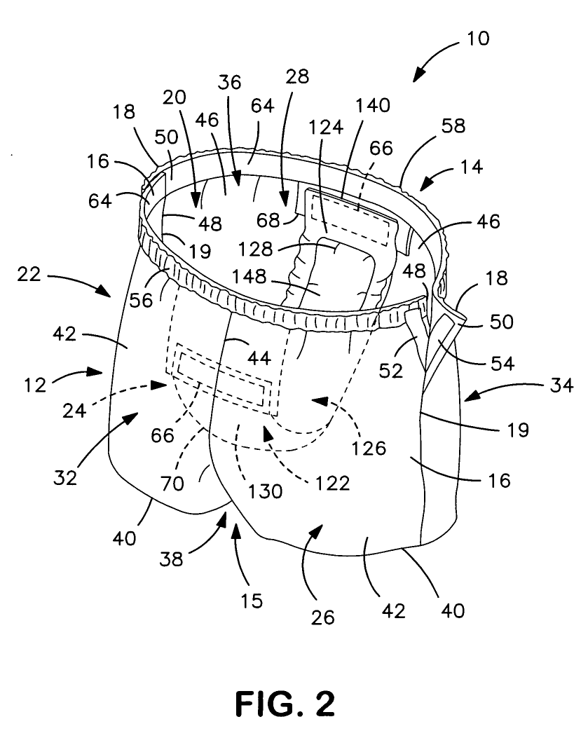

Referring now to the drawings, and in particular to FIGS. 1 and 2, an absorbent garment according to one embodiment of the present invention is indicated in its entirety by the reference numeral 10. The absorbent garment 10 is configured to be worn on a wearer's waist and generally has a front waist region, indicated generally at 12, a back waist region, indicated generally at 14 and a crotch region, indicated generally at 15. The front and back waist regions 12, 14 have respective side margins 16, 18 which are in particular embodiments attached to each other along side seams 19 of the garment to form a three-dimensional configuration of the garment during wear and having a waist opening, generally indicated at 20. As used herein, the term “seam” is intended to refer to a region along which two components are overlapped or otherwise in abutment with each other and may or may not be attached to each other.

As described further herein, the absorbent garment is suitably configured to resemble conventional clothing such as shorts (e.g., boxer shorts, gym shorts, running shorts, etc.), skirts, skorts (i.e., a combination of a skirt and a pair of shorts), swim trunks and the like, while providing the functions of conventional absorbent articles, such as taking in and retaining body exudates released by the wearer. The absorbent garment 10 comprises a garment shell, generally indicated at 22 and constructed to provide the desired resemblance of the garment to conventional clothing, and an absorbent assembly, generally indicated at 24, disposed within and releasably attached to the garment shell and constructed to take in and retain body exudates released by the wearer.

With particular reference to FIGS. 1 and 2, the garment shell 22 comprises a front panel assembly, which is generally indicated at 26, having laterally opposite side margins 48 and a back panel assembly, which is generally indicated at 28 in FIG. 2, having laterally opposite side margins 50. In the illustrated embodiment, the side margins 48 of the front panel assembly 26 broadly define the front side margins 16 of the absorbent garment 10 and the side margins 50 of the back panel assembly 28 broadly define the back side margins 18 of the absorbent garment. As will be described in further detail later herein, the side margins 48, 50 of the front and back panel assemblies 26, 28 of the garment shell 22 are overlapped and in particular embodiments are attached to each other to broadly define the side seams 19 of the absorbent garment 10, and to define the three-dimensional configuration of the garment shell during wear.

In its three-dimensional configuration as shown in FIGS. 1 and 2, the garment shell 22 has a front waist region 32 which at least in part defines the front waist region 12 of the absorbent garment 10, a back waist region 34 which at least in part defines-the back waist region 14 of the absorbent garment, and front and back waist ends, designated 56 and 58, respectively, which together generally define a waist opening 36 of the garment shell. In the illustrated embodiment, the garment shell 22 is configured to resemble a pair of shorts and thus further has a crotch region 38 extending longitudinally between and interconnecting the front waist region 32 and the back waist region 34 of the garment shell. The crotch region 38 of the garment shell 22 at least in part defines the crotch region 15 of the absorbent garment 10, and also in part defines leg openings 40 of the garment shell (broadly referred to herein as outer leg openings of the absorbent garment). However, it is understood that the crotch region 38 of the garment shell 22 may be omitted (so that the crotch region 15 of the absorbent garment 10 is defined solely by the absorbent assembly 24 as described later herein), such as where the garment shell is intended to resemble a skirt (in which case only one leg opening 40 of the garment shell is provided to accommodate both legs of the wearer), without departing from the scope of this invention.

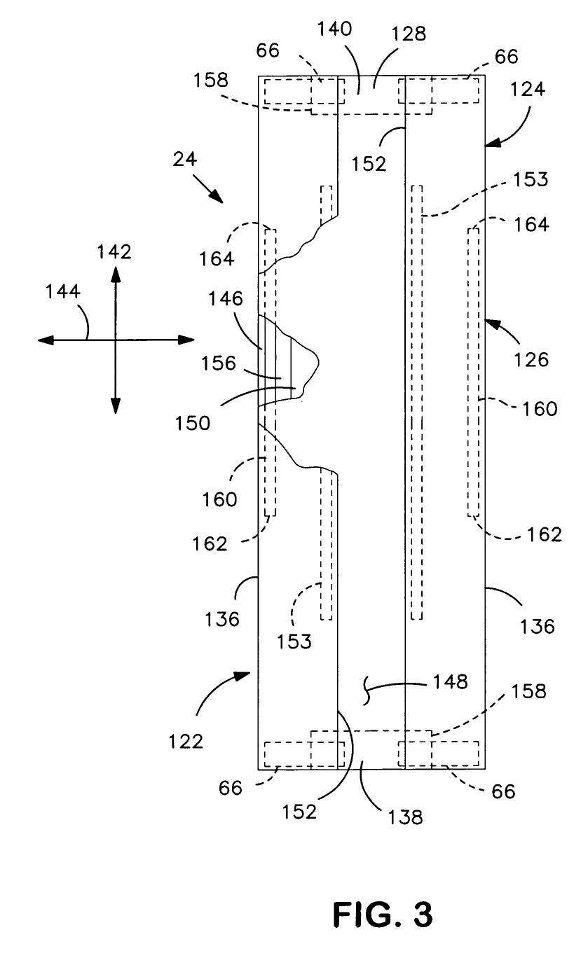

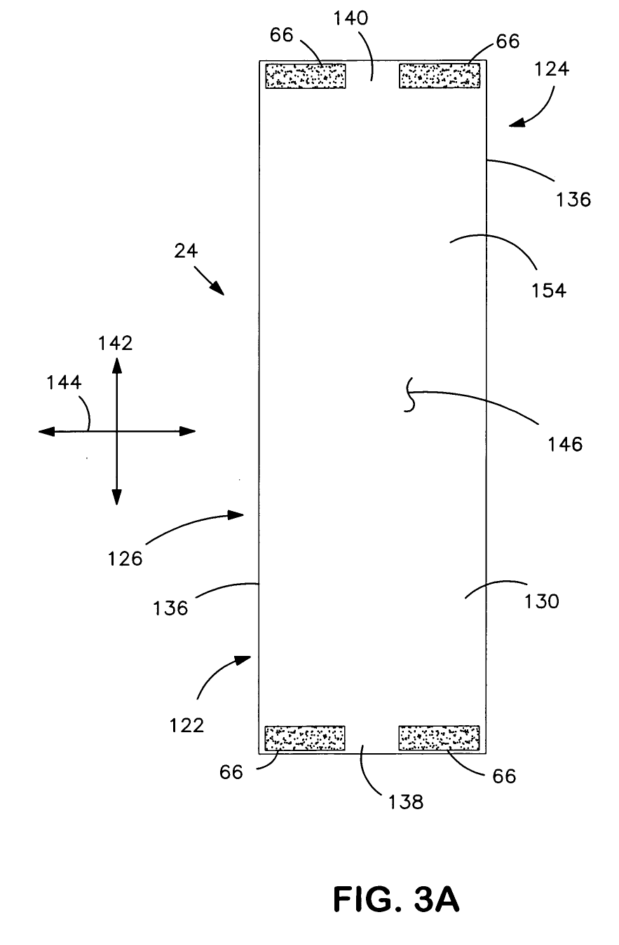

The front panel assembly 26 of the garment shell 22 comprises a pair of panel members 42 which are in particular embodiments permanently attached to each other, such as by ultrasonic bonding, pressure bonding, thermal bonding, adhesive bonding, stitching or other conventional attachment technique, along a central seam 44 extending longitudinally from the front waist region 32 to the crotch region 38 of the garment shell. The back panel assembly 28 comprises a pair of panel members 46 configured and permanently attached to each other in a manner similar to the panel members 42 of the front panel assembly 26 along a central seam 47 (FIG. 3) extending longitudinally from the back waist region 34 to the crotch region 38 of the garment shell 22. It is understood, however, that each of the front and back panel assemblies 26, 28 may be constructed of a single panel member (e.g., of unitary construction) without departing from the scope of this invention. Alternatively, the front and back panel members 42, 46 on one side of the garment shell 22 may be formed integrally at the crotch region 38 thereof so that no attachment of the panel members is necessary at the leg openings.

The panel members 42, 46 of the front and back panel assemblies 26, 28 of the garment shell 22 can be constructed of any suitable material, and more suitably a material that provides a generally cloth-like texture. The panel members 42, 46 are, in particular embodiments, constructed of a material which is relatively durable so that the garment shell 22 can be re-used through multiple replacements of the absorbent assembly. It is also contemplated that the panel members 42, 46 can, but need not necessarily be, constructed of a material suitable for laundering to permit laundering of the garment shell. Such materials may include knit fabrics such as stretch knit, fleece knit, herringbone knit, jersey knit, and raschel knit; and woven fabrics such as broadcloth, twill, percale, poplin, muslin, cambric, chino, flannel, silks and woolens.

In yet another alternative embodiment, the garment shell is intended to be disposable after a single or several uses. As an example, the panel members 42, 46 may be constructed from natural and/or synthetic sources and may be constructed in any suitable manner including, but not limited, to nonwovens such as spunbond webs, meltblown webs, spunbond film laminates, bonded carded webs, spunlace webs, hydroentangled webs, and needlepunched fabrics.

For instance, in one embodiment, the garment shell comprises a laminate material containing a spunbond web. For instance, the laminate may comprise a film or a meltblown layer laminated in between two opposite spunbond layers. The middle layer may be included into the laminate so as to hide the absorbent assembly and not make the absorbent assembly visible through the laminate. In one embodiment, for instance, the middle layer may comprise a meltblown web made from polyethylene alone or in combination with other polymers.

The spunbond layers laminated to the middle layer may be made from bicomponent filaments. For instance, the spunbond layers may contain bicomponents filaments having a sheath/core arrangement. The core may be made from polypropylene alone or in combination with other polymers. The sheath, on the other hand, may comprise a polyethylene polymer alone or in combination with other polymers.

The basis weight of each of the layers and of the laminate may be adjusted in order for the material to have the desired degree of softness, drape and opacity. In general, for instance, the laminate may have an overall basis weight of from about 1 osy to about 3 osy, such as from about 1 osy to about 1.5 osy. When the laminate comprises a spunbond/meltblown/spunbond laminate, the meltblown layer, for instance, may have a basis weight of from about 0.1 osy to about 1 osy, such as from about 0.2 osy to about 0.5 osy. The spunbond facings account for the remaining basis weight and both outer layers may have the same basis weight if desired.

If desired, the laminate material may include a bond pattern, such as a ribknit bond pattern. The bond pattern not only serves to attach the different layers together, but also increases the opacity of the material and gives the material a cloth-like look.

In order to improve the aesthetics of the garment, the garment shell may also include various colors and designs. For instance, solid colors as well as printed designs may be applied to the exterior surface of the garment shell. As just an example, the outer shell may have a pink color that includes butterfly and flower designs, or may be blue in color and include sports designs.

As described above, garment shells made in accordance with the present invention are configured to have carefully controlled friction properties when rubbed against the absorbent assembly or against an adjacent material, such as a bed sheet. In one embodiment, for instance, the coefficient of friction between the garment shell and the absorbent assembly should be at least about 15% less than the coefficient of friction between an exterior surface of the garment shell and a standard bed sheet material. In this regard, various techniques may be used in order to carefully control the friction properties of the outer shell.

For example, the bonding pattern described above may be only applied to the exterior surface of the garment shell thus creating a relatively rough exterior surface and a relatively smooth interior surface. Further, controlling the density of the bonding pattern also may have an effect on the coefficient of friction. In general, a less dense bonding pattern generally increases the coefficient of friction against an opposite material.

Another technique to control the coefficient of friction of the garment shell is to select the polymers that form the sheath of the bicomponent filaments. For example, polyethylene tends to produce a surface having a lower coefficient of friction than when using polypropylene. Further, by incorporating other polymers into the sheath, the coefficient of friction may be adjusted.

Still another technique for adjusting the coefficient of friction of the garment shell is to increase or decrease the size of the spunbond filaments. In general, larger diameter filaments will create a material having a larger coefficient of friction. In general, for instance, the diameter of meltblown fibers can vary from about 1 micron to about 5 microns. Spunbond fibers, on the other hand, may vary in diameter of from about 15 microns to about 20 microns.

In addition to the above techniques, the basis weight of the materials may also be used to adjust and control the coefficient of friction of the garment shell. In general, for instance, increasing the basis weight of a nonwoven web will generally increase the coefficient of friction of that material. Thus, in one embodiment, the exterior surface of the garment shell may contain a spunbond web that has a heavier basis weight than the spunbond web that is positioned on the interior of the garment shell.

Referring back to FIG. 2, the front and back panel assemblies 26, 28 of the garment shell 22 can be releasably attached to each other at the respective side margins 48, 50 of the panel assemblies. For example, in the illustrated embodiment a fastening component 52 is attached to each side margin 48 of the front panel assembly 26 and is adapted for refastenable engagement with a complementary fastening component 54 attached to each respective side margin 50 of the back panel assembly 28. Although the garment shell 22 as illustrated in FIG. 2 has the side margins 50 of the back panel assembly 28 overlapping the side margins 48 of the front panel assembly 26 upon releasable attachment, the garment shell can instead be configured so that the side margins of the front panel assembly overlap the side margins of the back panel assembly for releasable attachment.

The fastening components 52, 54 can comprise any refastenable fasteners suitable for garments, such as adhesive fasteners, cohesive fasteners, mechanical fasteners, or the like. In particularly suitable embodiments, the fastening components 52, 54 comprise mechanical fastening elements provided by interlocking geometric shaped materials such as hooks, loops, bulbs, mushrooms, arrowheads, balls on stems, male and female mating components, buckles, snaps, or the like. For example, in the illustrated embodiment the fastening components 52 comprise hook fasteners and the fastening components 54 comprise complementary loop fasteners arrayed so that the hook fasteners face generally away from the wearer. Alternatively, the fastening components 52 may comprise loop fasteners and the fastening components 54 may comprise complementary hook fasteners.

The fastening components 52, 54 extend along the respective side margins 48, 50 of the front and back panel assemblies 26, 28 generally from the waist ends 56, 58 of the panel assemblies to a position intermediate the waist ends and the leg openings 40 of the garment shell so that the absorbent garment side margins 16, 18 are releasably attached to each other along upper segments of the side seams 19. As an example, the fastening components 52, 54 suitably extend from the front and back waist ends 56, 58 of the garment shell 22 along the side margins 48, 50 thereof in the range of about 30 percent to about 90 percent of the length of the side margins (broadly, about 30 percent to about 90 percent of the length of the side seams 19 of the absorbent garment 10). However, it is understood that the fastening components 52, 54 may be longer or shorter without departing from the scope of this invention. Thus, in the illustrated embodiment, only a portion of the side seams 19 of the absorbent garment are releasably attached.

The segment of the garment shell 22 along which the side margins 48, 50 are not releasably attached (e.g., extending from the bottom of the fastening components 52, 54 to the leg openings 40 of the garment shell) are suitably free from any form of attachment. In such an embodiment, the non-refastenable portion of the side seams 19 of the absorbent garment 10 are referred to as being open and the side margins 16, 18 thereof are referred to as being unattached.

Alternatively, the side margins 48, 50 of the front and back assemblies 26, 28 of the garment shell 22 may be non-refastenably (e.g., frangibly or permanently) attached to each other along the portion of side margins extending from the bottom of the fastening components to the leg openings 40, such as by adhesive, by thermal, ultrasonic, or pressure bonding, or by other suitable attachment techniques.

It is also contemplated that the fastening components 52, 54 may instead extend from the leg openings 40 of the garment shell partially up along the side margins 48, 50 of the front and back panel assemblies 26, 28 (e.g., so that only a lower segment of the side seams 19 of the absorbent garment are refastenable). The side margins 48, 50 extending from the tops of the fastening components 52, 54 to the waist ends 56, 58 of the garment shell 22 may be non-refastenably (e.g., frangibly or permanently) attached to each other in the manner described previously.

In other embodiments, the fastening components 52, 54 may extend the entire length of the side margins 48, 50 of the front and back panel assemblies 26, 28 of the garment shell 22 (e.g., such that the side seams 19 of the absorbent garment 10 are refastenable along their full length). Also, while the fastening components 52, 54 are illustrated as being continuous along each respective side margin 48, 50, it is understood that two or more fastening components may be attached to each respective side margin in spaced relationship along the side margin without departing from the scope of this invention.

It is further contemplated that the side margins 48, 50 of the garment shell 22 may instead be permanently or frangibly (e.g., non-refastenably) attached along all or part of the full length thereof whereby no portions of the side margins are refastenable. It is also understood that the garment shell 22 may be formed to omit the side margins 48, 50 thereof, such as by integrally forming the respective front and back panel members 42, 46 on each side of the shell.

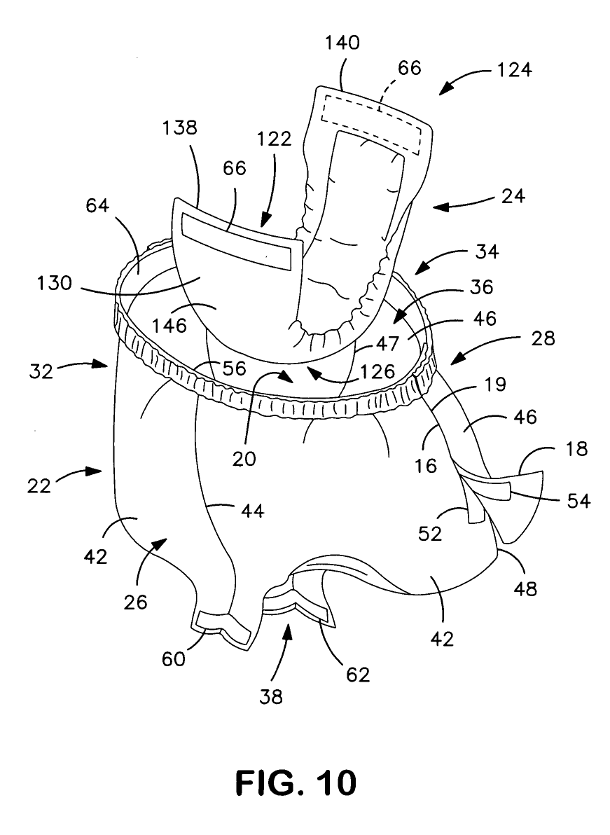

In addition to the front and back panel assemblies 26, 28 of the garment shell 22 being releasably attached to each other at the respective side margins 48, 50 thereof, or as an alternative thereto, it is contemplated that the panel assemblies may be releasably attached to each other at the crotch region 38 of the garment shell to allow the garment shell to be unfastened at the crotch region and pulled up relative to the absorbent assembly 24 for inspecting or otherwise replacing the absorbent assembly. For example, fastening components (not shown in FIG. 2 but indicated at 60, 62 in FIG. 10) may be attached to the front and back panel assemblies 26, 28 of the garment shell 22 generally at the crotch region 38 thereof to permit releasable attachment of the panel assemblies at the crotch region.

To further enhance the appearance of the absorbent garment 10 as well as the fit of the absorbent garment on the wearer's waist, one or more elastic members 64 (e.g., waistband elastics) can be operatively joined to the front and back panel assemblies 26, 28 generally at the respective waist ends 56, 58 thereof. For example, as best seen in FIG. 2, an elastic member 64 can be operatively joined to the front waist end 56 of the garment shell 22 on laterally opposite sides of the absorbent assembly 24. Another elastic member 64 can be operatively joined to the back waist end 58, also on laterally opposite sides of the absorbent assembly 24. The elastic members 64 can be operatively joined to the garment shell 22 while in a stretched condition so that upon retraction the elastic members gather the garment shell at the front and back waist ends 56, 58 to provide a gathered appearance and to further provide an elastic fit of the absorbent garment on the wearer's waist. Alternatively, it is contemplated that multiple elastic members (not shown) may be attached to each waist end 56, 58 of the garment shell 22 and extend laterally across all or only a portion of the width of the respective waist end without departing from the scope of this invention.

With further reference to FIGS. 2-4, the absorbent assembly 24 comprises a front waist region 122, a back waist region 124, a crotch region 126 interconnecting the front and back waist regions, an inner surface 128 configured for contiguous relationship with the wearer, and an outer surface 130 opposite the inner surface. The front waist region 122 comprises the portion of the absorbent assembly which, when the absorbent garment 10 is worn, is positioned on the front of the wearer while the back waist region 124 comprises the portion of the absorbent assembly which is positioned on the back of the wearer. The crotch region 126 of the absorbent assembly 24 comprises the portion of the assembly which is positioned between the legs of the wearer and covers the lower torso of the wearer. With additional reference to FIG. 3, the absorbent assembly 24 also has laterally opposite side edges 136 and longitudinally opposite waist ends, respectively designated herein as front waist end 138 and back waist end 140.

The absorbent assembly 24 is suitably “disposable,” which as used herein refers to articles that are intended to be discarded after a limited period of use instead of being laundered or otherwise restored for reuse. However, it is contemplated that the absorbent assembly may be re-useable and remain within the scope of this invention. By way of illustration only, various materials and methods for constructing the absorbent assembly 24 are disclosed in PCT Patent Application WO 00/37009 published Jun. 29, 2000 by A. Fletcher et al; U.S. Pat. No. 4,940,464 issued Jul. 10, 1990 to Van Gompel et al.; and U.S. Pat. No. 5,766,389 issued Jun. 16, 1998 to Brandon et al., which are incorporated herein by reference.

The absorbent assembly 24, as is illustrated in FIGS. 3 and 3A detached from the garment shell 22 and in a laid flat configuration. The absorbent assembly 24, in this embodiment, is illustrated as being rectangular in shape, and has a longitudinal axis 142 and a transverse, or lateral axis 144. It is understood that the absorbent assembly 24 may be other than rectangular, such as hourglass-shaped, T-shaped, I-shaped or other suitable shape without departing from the scope of this invention. Referring to FIG. 3, the absorbent assembly 24 comprises an outer cover 146, a bodyside liner 148 in superposed relationship with the outer cover, an absorbent body 150 disposed between the outer cover and the bodyside liner, and a pair of laterally spaced containment flaps 152 configured to inhibit the transverse flow of body exudates on the liner to the side edges 136 of the absorbent assembly.

The outer cover 146 of the absorbent assembly 24 can, but need not, comprise a material which is substantially liquid impermeable, and can be stretchable or non-stretchable. The outer cover 146 can be made from a single layer of material or can comprise a multi-layered laminate. As used herein, the term “stretchable” refers to a material that may be extensible or elastic. That is, the material may be extended, deformed or the like, without breaking, and may or may not significantly retract after removal of an extending force. As used herein, the term “elastic” refers to that property of a material where upon removal of an elongating force, the material is capable of recovering to substantially its unstretched size and shape, or the material exhibits a significant retractive force. The term “extensible” refers to that property of a material where upon removal of an elongating force, the material experiences a substantially permanent deformation, or the material does not exhibit a significant retractive force.

In one embodiment, the outer cover 146 comprises a multi-layered laminate structure in which at least one of the layers is liquid impermeable. For instance, referring to FIGS. 3 and 3A, the outer cover 146 can include a liquid permeable outer layer 154 and a liquid impermeable inner layer 156 which are suitably joined together by a laminate adhesive, ultrasonic bonds, pressure bonds, thermal bonds, or the like. Suitable laminate adhesives, which can be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A.

In one particular embodiment, for instance, the outer layer 154 of the outer cover 146 may comprise a spunbond web, while the liquid impermeable inner layer 156 may comprise a polymer film. The polymer film may comprise, for instance, polypropylene, polyethylene, copolymers thereof or mixtures thereof. The spunbond web, on the other hand, may have a relatively low basis weight, such as from about 10 gsm to about 30 gsm. The spunbond web may provide the outer cover 146 with a cloth-like texture and appearance.

In general, the outer cover 146 may be constructed in a manner so as to have a suitable coefficient of friction, especially when tested against the interior surface of the garment shell. In order to control the coefficient of friction of the outer cover 146, the above techniques described with respect to the garment shell may be used. For instance, the filament diameter, the basis weight, and the materials used to form the spunbond layer of the outer cover can be varied as desired in order to arrive at a target coefficient of friction.

Alternatively, the outer cover 146 may not contain a spunbond web but, instead, may have an exterior surface comprising the polymer film. In general, films are going to have a lower coefficient of friction than nonwoven webs. Thus, it may be desired to have a film serve as the outer surface of the outer cover 146 for minimizing the coefficient of friction between the outer cover 146 and the garment shell.

In still another embodiment of the present invention, the outer cover 146 may comprise a stretchable laminate, such as a spunbond film laminate or the like.

Alternatively, the outer cover 146 may comprise a single layer of liquid impermeable material. The liquid impermeable material can permit vapors to escape from the interior of the disposable absorbent article, while still preventing liquids from passing through the outer cover 146. For example, the outer cover 146 may be constructed of a microporous polymer film or a nonwoven fabric that has been coated or otherwise treated to impart a desired level of liquid impermeability. One such microporous film is a PMP-1 film material commercially available from Mitsui Toatsu Chemicals, Inc., Tokyo, Japan, or an XKO-8044 polyolefin film commercially available from 3M Company, Minneapolis, Minn. U.S.A. The single layer outer cover 146 may also be embossed and/or matte finished to provide a more cloth-like appearance.

The liquid permeable bodyside liner 148 is illustrated as overlying the outer cover 146 and absorbent body 150, and may but need not have the same dimensions as the outer cover 146. The bodyside liner 148 is desirably compliant, soft feeling, and non-irritating to the wearer's skin. Further, the bodyside liner 148 can be less hydrophilic than the absorbent body 150, to present a relatively dry surface to the wearer and to permit liquid to readily penetrate through the liner. Alternatively, the bodyside liner 148 can be more hydrophilic or can have essentially the same affinity for moisture as the absorbent body 150 to present a relatively wet surface to the wearer to increase the sensation of being wet. This wet sensation can be useful as a training aid. The hydrophilic/hydrophobic properties can be varied across the length, width and depth of the bodyside liner 148 and absorbent body 150 to achieve the desired wetness sensation or leakage performance.

The bodyside liner 148 can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics can be used for the bodyside liner 148. For example, the bodyside liner 148 can be composed of a meltblown or spunbonded web of polyolefin fibers. The bodyside liner can also be a bonded-carded web composed of natural and/or synthetic fibers. The bodyside liner 148 can be composed of a substantially hydrophobic material, and the hydrophobic material can, optionally, be treated with a surfactant or otherwise processed to impart a desired level of wettability and hydrophilicity. For example, the material can be surface treated with about 0.45 weight percent of a surfactant mixture comprising Ahcovel N-62 from Hodgson Textile Chemicals of Mount Holly, N.C., U.S.A. and Glucopan 220UP from Henkel Corporation of Ambler, Pa. in an active ratio of 3:1. The surfactant can be applied by any conventional means, such as spraying, printing, brush coating or the like. The surfactant can be applied to the entire bodyside liner 148 or can be selectively applied to particular sections of the bodyside liner, such as the medial section along the longitudinal center line.

One example of a suitable liquid permeable bodyside liner 148 is a nonwoven bicomponent web having a basis weight of about 27 gsm. The nonwoven bicomponent web can be a spunbond bicomponent web, or a bonded carded bicomponent web. Suitable bicomponent fibers include a polyethylene/polypropylene bicomponent fiber available from CHISSO Corporation, Osaka, Japan. In this particular bicomponent fiber, the polypropylene forms the core and the polyethylene forms the sheath of the fiber. Other fiber orientations are possible, such as multi-lobe, side-by-side, end-to-end, or the like.

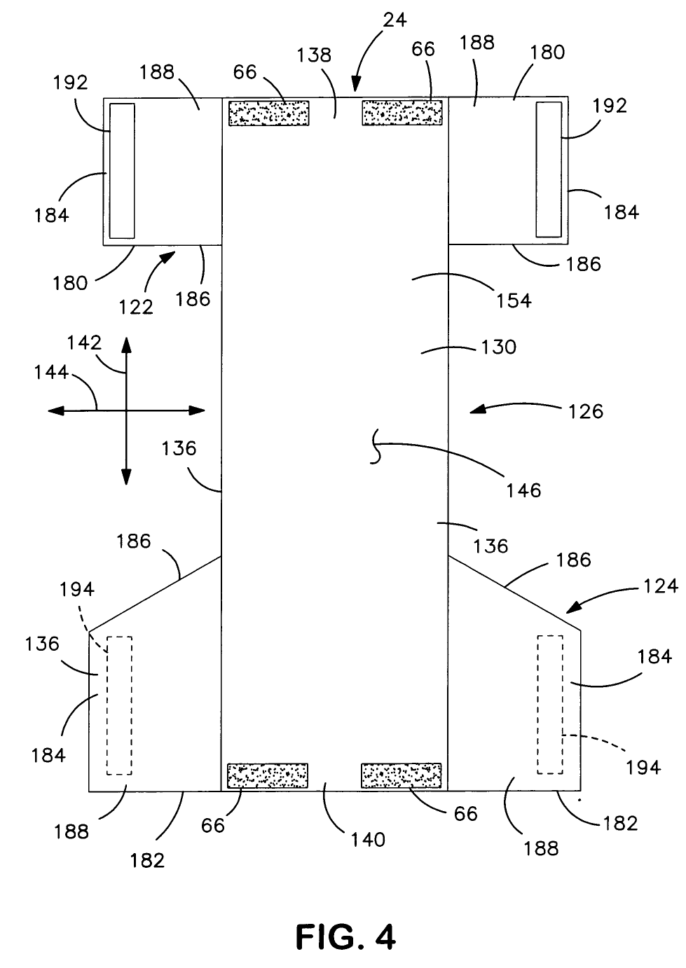

The absorbent body 150 (FIG. 4) is positioned between the outer cover 146 and the bodyside liner 148, which can be joined together by any suitable means such as adhesives, ultrasonic bonds, pressure bonds, thermal bonds, or the like. The absorbent body 150 can be any structure which is generally compressible, conformable, non-irritating to the child's skin, and capable of absorbing and retaining liquids and certain body wastes, and may be manufactured in a wide variety of sizes and shapes, and from a wide variety of liquid absorbent materials commonly used in the art. For example, the absorbent body 150 can suitably comprise a matrix of hydrophilic fibers, such as a web of cellulosic fluff, mixed with particles of a high-absorbency material commonly known as superabsorbent material. In a particular embodiment, the absorbent body 150 comprises a matrix of cellulosic fluff, such as wood pulp fluff, and superabsorbent hydrogel-forming particles. The wood pulp fluff can be exchanged with synthetic, polymeric, meltblown fibers or short cut homofil bicomponent synthetic fibers and natural fibers. The superabsorbent particles can be substantially homogeneously mixed with the hydrophilic fibers or can be nonuniformly mixed. The fluff and superabsorbent particles can also be selectively placed into desired zones of the absorbent body 150 to better contain and absorb body exudates. The concentration of the superabsorbent particles can also vary through the thickness of the absorbent body 150. Alternatively, the absorbent body 150 can comprise a laminate of fibrous webs and superabsorbent material, a foam or other suitable web construction.

Suitable superabsorbent materials can be selected from natural, synthetic, and modified natural polymers and materials. The superabsorbent materials can be inorganic materials, such as silica gels, or organic compounds, such as crosslinked polymers, for example, sodium neutralized polyacrylic acid. Suitable superabsorbent materials are available from various commercial vendors, such as BASF Corporation, Charlotte, N.C., U.S.A., and Stockhausen GmbH & Co. KG, D-47805 Krefeld, Federal Republic of Germany. Typically, a superabsorbent material is capable of absorbing at least about 10 times its weight in water, and suitably is capable of absorbing more than about 25 times its weight in water.

In one embodiment, the absorbent body 150 comprises a blend of wood pulp fluff and superabsorbent material. One suitable type of pulp is identified with the trade designation CR1654, available from U.S. Alliance, Childersburg, Ala., U.S.A., and is a bleached, highly absorbent sulfate wood pulp containing primarily soft wood fibers and about 16 percent hardwood fibers. In general, the superabsorbent material is present in the absorbent body 150 in an amount of from 0 to about 90 weight percent based on total weight of the absorbent assembly. The absorbent body 150 may or may not be wrapped or encompassed by a suitable wrap, such as a meltblown wrap or cellulosic tissue wrap, that aids in maintaining the integrity and/or shape of the absorbent assembly during use.

The containment flaps 152 are located generally adjacent to the side edges 136 of the absorbent assembly 24, and can extend longitudinally along the entire length of the absorbent assembly 24 as shown in FIG. 3 or only partially along the length of the absorbent assembly. Flap elastic members 153 (FIG. 3) can be operatively joined with the containment flaps 152 in a suitable manner as is well known in the art, such as by adhering the elastic members to the flaps while the elastic members are in a stretched condition so that the flaps are biased by the elastic members to a longitudinally gathered configuration. The elasticized containment flaps 152 can define a partially unattached distal edge (not shown), unattached to the liner 148, which assumes an upright configuration in at least the crotch region 126 of the absorbent assembly 24 during wear to form a seal (e.g., an elastic fit) against the wearer's body. Suitable constructions and arrangements for the containment flaps 152 are generally well known to those skilled in the art and are described in U.S. Pat. No. 4,704,116 issued Nov. 3, 1987 to Enloe, which is incorporated herein by reference. It is understood, however, that the containment flaps 152 may be omitted without departing from the scope of this invention.

To further enhance the fit of the absorbent garment 10 on the wearer and to further inhibit leakage of body exudates, the absorbent assembly can also have waist elastic members 158 (FIG. 3) and leg elastic members 160 (FIG. 3), as are known to those skilled in the art. The waist elastic members 158 can be operatively joined to the absorbent assembly 24 at the waist ends 138 and 140, such as by attaching the elastic members to the outer cover 146 and/or the bodyside liner 148 while the elastic members are in a stretched condition, so that upon retraction the elastic members gather the absorbent assembly at the waist ends to provide an elastic fit against the wearer's waist. In the illustrated embodiment the elastic members 158 which are operatively joined to the absorbent assembly 24, and the elastic members 64 which are operatively joined to the garment shell 22 on laterally opposite sides of the absorbent assembly, together provide an elastic fit of the absorbent garment 10 against substantially the entire waist of the wearer. The elastic members 158 are shown in FIG. 3 as extending only partially across the respective front and back waist ends 138, 140 of the absorbent assembly 24. It is understood, however, that the elastic members 158 may extend laterally across the full width of the absorbent assembly 24 at one or both waist ends 138, 140 without departing from the scope of this invention.

The leg elastic members 160 can be operatively joined to the outer cover 146 and/or the bodyside liner 148 and extend longitudinally adjacent the opposite side edges 136 generally through the crotch region 126 of the absorbent assembly 24. Each leg elastic member 160 has, in particular embodiments, a front terminal point 162 and a back terminal point 164, which represent the longitudinal ends of the elastic gathering caused by the leg elastic members.

The flap elastic members 153, the waist elastic members 158 (as well as the elastic members 64 operatively joined with the garment shell 22), and the leg elastic members 160 can be formed of any suitable elastic material. As is well known to those skilled in the art, examples of suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic retractive forces are imparted to the substrate.

The absorbent assembly 24 can also incorporate other materials or components designed primarily to receive, temporarily store, and/or transport liquid along the mutually facing surface with the absorbent body 150, thereby maximizing the absorbent capacity of the absorbent assembly. For example, one suitable additional component is commonly referred to as a surge layer (not shown). Surge layers are generally well known in the art as being constructed to quickly collect and temporarily hold liquid surges, and to transport the temporarily held liquid to the absorbent body 150.

Various woven and non-woven fabrics can be used to construct the surge layer. For example, the surge layer may be a layer made of a meltblown or spunbond web of synthetic fibers, such as polyolefin fibers. The surge layer may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge layer may be composed of a substantially hydrophobic material, and the hydrophobic material may optionally be treated with a surfactant or otherwise processed to impart a desired level of wettability and hydrophilicity.

Examples of materials suitable for the surge layer are set forth in U.S. Pat. No. 5,486,166 issued Jan. 23, 1996 in the name of C. Ellis et al.; U.S. Pat. No. 5,490,846 issued Feb. 13, 1996 in the name of Ellis et al.; and U.S. Pat. No. 5,364,382 issued Nov. 15, 1994 in the name of Latimer et al., the disclosures of which are hereby incorporated by reference in a manner consistent with the present document.

Referring to FIGS. 1-2 and 5-8, the absorbent assembly 24 is attached to the garment shell 22. The front and back waist regions 122, 124 of the absorbent assembly 24 are attached to the garment shell 22 generally at the front and back waist regions 32, 34 thereof, respectively. As described in more detail below, the absorbent 24 assembly may be permanently attached, removably attached, or refastenably attached to the garment shell 22. Further, the attachment may be directly to the garment shell 22, or indirectly by way of an intervening element or elements.

Referring to the exemplary embodiments shown in FIGS. 1-2, 5-8, and 9, the absorbent garment 10 of the present invention includes at least one inner attachment member 70 disposed at one or both of the garment shell waist regions 32 and 34. The absorbent assembly 24 is refastenably attached to the inner attachment member 70 to permit detachment, adjustment, and reattachment of the absorbent assembly. In this way, the position of the absorbent assembly 24 within the garment 10 can be adjusted to fit users of different sizes. Further, in certain embodiments as will be described shortly, the absorbent assembly 24 can be removed altogether from the absorbent garment 10, permitting it to be replaced, adjusted, or even omitted to allow the garment shell 22 to be worn without an absorbent assembly 24.

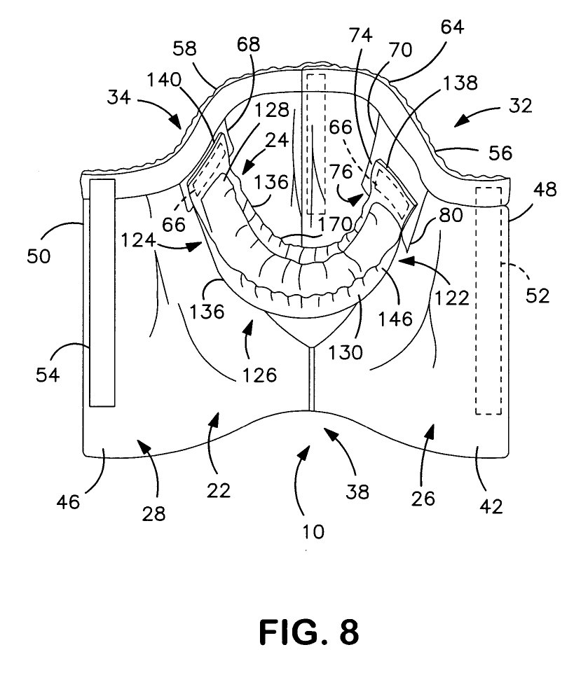

Referring to the exemplary embodiments shown in FIGS. 1, 2, and 8, the front and back waist end 138, 140 of the absorbent assembly 24 are refastenably attached to the inner attachment member 70 and the garment shell back waist region 34, respectively. At least one fastening component 66 is attached to the outer cover 146 of the absorbent assembly 24 generally at the front waist end 138 thereof. An inner attachment member 70 is disposed at the inner surface of the front waist region 32 of the garment shell 22 for refastenable attachment to the fastening components 66 at the front waist end 138 of the absorbent assembly 24. One or more additional fastening components 66 can be attached to the outer cover 146 of the absorbent assembly 24 generally at the back waist end 140 thereof with corresponding fastening components 68 (FIG. 8) being attached to the inner surface of the back waist end 58 of the garment shell 22 for releasable attachment to the fastening components 66 at the back waist end 140 of the absorbent assembly 24.

The fastening components 66, 68 can comprise any refastenable fasteners suitable for garments as described previously herein, such as adhesive fasteners, cohesive fasteners, mechanical fasteners, or the like. In particularly suitable embodiments, the fastening components 66, 68 comprise mechanical fastening elements provided by interlocking geometric shaped materials such as hooks, loops, bulbs, mushrooms, arrowheads, balls on stems, male and female mating components, buckles, snaps, or the like.

With the absorbent assembly 24 refastenably attached to the garment shell 22, the elasticized side edges 136 of the absorbent assembly 24 generally define laterally opposite elastic leg openings 170 (FIGS. 1, 5, 7, 8, and 9) of the absorbent assembly (broadly, inner leg openings of the absorbent garment 10) whereat the absorbent assembly 24 provides an elastic fit against at least part of the wearer's legs. The leg openings 40 of the garment shell 22 broadly define outer leg openings of the absorbent garment 10, separate (e.g., discrete) from the absorbent assembly leg openings 170, whereat the absorbent garment hangs generally loose about the wearer's legs.

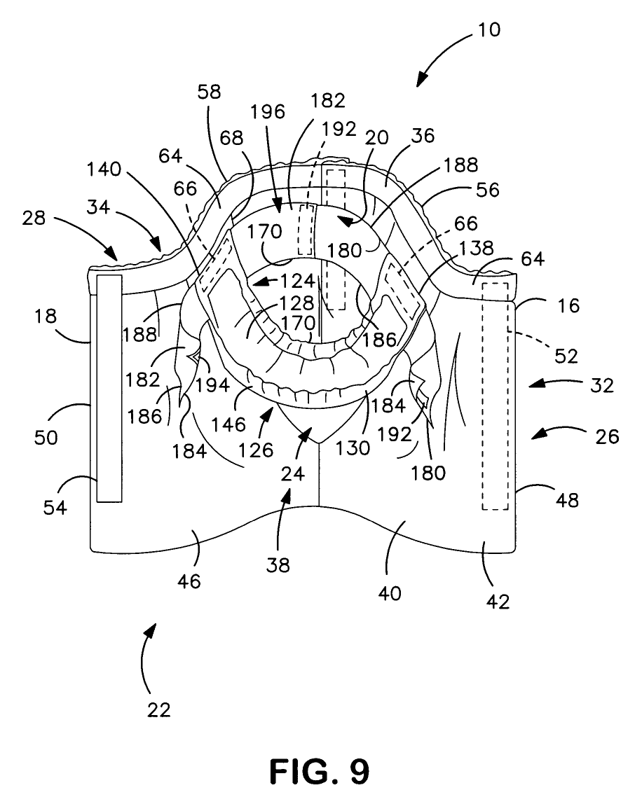

In an alternative embodiment of the absorbent assembly 24 as shown in FIG. 4 (as well as in another alternative embodiment of an absorbent garment 10 shown in FIG. 9 and described further below), the absorbent assembly 24 may be a brief-style absorbent assembly such as children's training pants, swim pants or child or adult enuresis or incontinence products which are configured for wearing about the full waist of the wearer. More particularly, in addition to the components illustrated in FIG. 3 and described previously herein, the absorbent assembly 24 illustrated in FIGS. 4 and 9 further comprises front and back side panels, designated 180 and 182, respectively, disposed generally on each side of the absorbent assembly 24 at the respective front and back waist regions 122, 124 of the absorbent assembly and extending transversely outward therefrom. The side panels 180, 182 may be attached to the bodyside liner 148 and/or to the outer cover 146 of the absorbent assembly 24 by adhesive, or by thermal or ultrasonic bonding, or by other suitable attachment techniques known to those skilled in the art. Alternatively, the side panels 180, 182 can be formed as an integral portion of a component of the absorbent assembly 24. For example, the side panels 180, 182 can comprise a generally wider portion of the outer cover 146, the bodyside liner 148, and/or another component of the absorbent assembly 24.

The front and back side panels 180, 182 have respective outer edges 184 which broadly define the side edges of the absorbent assembly 24 at the front and back waist regions 122, 124 thereof. The side panels 180, 182 also have respective leg end edges 186 disposed toward the longitudinal center of the absorbent assembly 24, and respective waist end edges 188 which further define the respective front or back waist end 138, 140 of the absorbent assembly 24. The leg end edges 186 of the back side panels 182 can be curved and/or angled (FIG. 4) relative to the transverse axis 144 to provide a better fit of the absorbent assembly 24 about the wearer's legs. However, it is understood that the leg end edges 186 of the front side panels 180 may additionally, or alternatively, be curved or angled, or none of the leg end edges may be curved or angled, without departing from the scope of this invention.

The side panels 180, 182 suitably comprise a stretchable material, and more suitably an elastic material, capable of stretching in a direction generally parallel to the transverse axis 144 of the absorbent assembly 24. Suitable elastic materials, as well as one process of incorporating elastic side panels into brief-style absorbent assemblies, are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. In particular embodiments, the elastic material may comprise a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference. Alternatively, the side panel material may comprise other woven or nonwoven materials, such as those described above as being suitable for the outer cover 146 or bodyside liner 148; mechanically pre-strained composites; stretchable but inelastic (e.g., extensible) materials; or non-stretchable materials.

Still referring to FIGS. 4 and 9, the absorbent assembly 24 of this embodiment further comprises laterally spaced first fastening components 192 attached to the front side panels 180 generally at the outer edges 184 thereof and complementary second fastening components 194 attached to the back side panels 182 generally at the outer edges thereof and adapted for refastenable engagement with the first fastening components to releasably attach the side panels together to thereby define a three-dimensional configuration of the absorbent assembly that can be worn about the waist of the wearer. The fastening components 192, 194 can comprise any of the refastenable fasteners previously described herein as being suitable for absorbent garments, such as adhesive fasteners, cohesive fasteners, mechanical fasteners, or the like. In particular embodiments the fastening components 192, 194 comprise mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like.

In the embodiment representatively illustrated in FIG. 9, the side panels 180, 182, when attached to each other to define the three-dimensional configuration of the absorbent assembly 24, define, together with the front and back waist ends 138, 140 of the absorbent assembly, a waist opening 196 of the absorbent assembly separate from the waist opening 36 of the garment shell 22. The side edges 136 of the absorbent assembly 24, including the leg end edges 186 of the side panels 180, 182, define the elastic leg openings 170 (broadly, the inner leg openings of the absorbent garment 10) of the absorbent assembly 24 about which the absorbent assembly provides an elastic fit against the wearer's leg. The attached side margins 48, 50 of the front and back panel assemblies 26, 28 of the garment shell 22 broadly define the side margins 16, 18 of the absorbent garment 10 which are, in particular embodiments, releasably attached along all or part of the side seams 19 of the absorbent garment. As in the embodiment of FIG. 2, the leg openings 40 of the garment shell 22 shown in FIG. 9 (when the garment shell is in its fully fastened configuration) broadly define outer leg openings of the absorbent garment 10 separate (e.g., discrete) from the leg openings 170 of the absorbent assembly whereat the absorbent garment hangs generally loose about the wearer's legs.

In the embodiment shown in FIG. 9, the front and side panels 180 and the back side panels 182 are connected to each side of the absorbent assembly 24 at the front and back waist regions. In an alternative embodiment, however, the front side panels 180 and the back side panels 182 may be replaced by a continuous elastic band that defines the waist opening 196 of the absorbent assembly. The elastic band may be positioned along and adjacent to the front waist end 138 and back waist end 140 of the absorbent assembly or, alternatively, may be positioned more towards the crotch region 126 of the absorbent assembly. When incorporated into the absorbent assembly, the continuous waist band may be permanently bonded to the garment shell or may be releasably attached to the garment shell as desired. In one embodiment, the waist band may include a frangible line of weakness in the lateral direction, such as a perforation line, that allows the absorbent assembly to be disconnected from the absorbent garment 10.

As representatively illustrated in FIGS. 1-2 and 5-8, an inner attachment member 70 is disposed at the garment shell front waist region 32. The inner attachment member 70 defines a fastening surface 74 refastenably engageable with the fastening components 66 present on the absorbent assembly 24. For example, the absorbent assembly 24 can be refastenably attached to the inner attachment member 70 by fastening components 66 attached to the outer surface of front and back waist ends 138, 140 of the absorbent. In the illustrated embodiment, the fastening components 66 attached to the front and back waist ends 138, 140 of the absorbent assembly 24 are hook fasteners. In such an embodiment, the inner attachment member 70 includes a suitable loop material for releasable attachment to the hook fasteners 66 of the absorbent assembly 24.

The inner attachment member can define a fastening surface 74 suitable for engagement with fasteners 66 over its entire surface, or over only a portion of its surface. For example, if fasteners 66 are hook-type fasteners, the inner attachment member 70 may integrally comprise a material which functions as a complementary, loop-type engaging surface. In an alternative embodiment (not shown), the inner attachment member 70 may include one or more complementary mating fastening components bonded to the inner attachment member 70 for refastenable attachment to the fastening component 66 attached to the front waist end 138 of the absorbent assembly 24. In such an embodiment, the fastening components attached to the inner attachment member 70 and to the absorbent assembly 24 may comprise any of the fastening components previously described herein as being suitable for absorbent garments. In particular embodiments, the entire fastening surface 74 of the inner attachment member 70 is a variable-location fastening surface, such that the fastening component 66 can be releaseably positioned anywhere along the entire length of the inner attachment member 70.

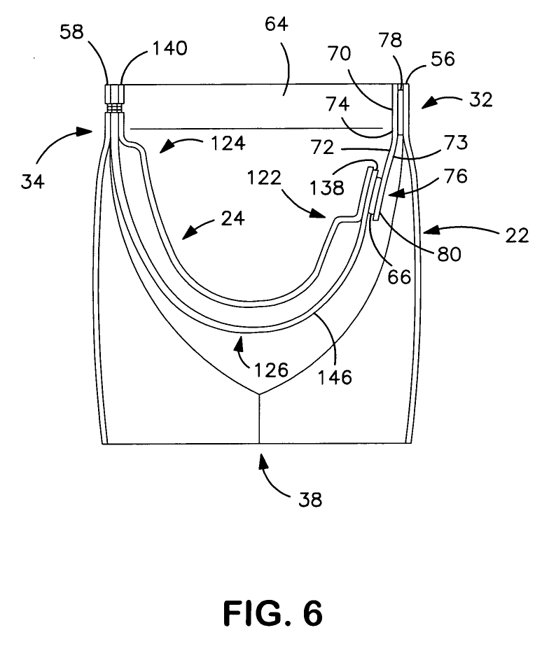

In particular embodiments, the inner attachment member 70 is permanently attached to the garment shell 22, such as by adhesive, by thermal, pressure, or ultrasonic bonding, or by other suitable attachment technique. For example, the inner attachment member can be attached by an adhesive 78, as representatively illustrated in FIG. 6. Alternatively, the inner attachment member 70 can be releasably attached to the garment shell 22, such as by fastening components attached to the inner surface of the garment shell generally at the front waist end 56 thereof (not shown). In yet another embodiment, the inner attachment member 70 may be formed integrally with the garment shell 22, such that the inner attachment member 70 defines at least part of the inner surface 27 of the front panel assembly 26 of the garment shell 22, and such that the at least part of the inner surface 27 of the garment shell 22 defines the fastening surface 74.

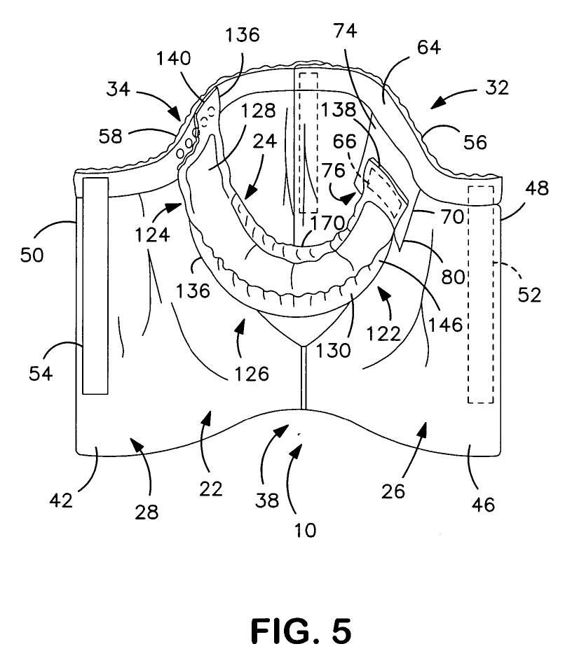

The inner attachment member 70 has a length dimension generally parallel to the longitudinal axis 142. In particular embodiments, at least a portion of the inner attachment member 70 is not adhered to the garment shell 22, so as to define an unadhered flap portion 80. For example, as can be seen in the embodiment shown in FIGS. 5-8, the inner attachment member 70 can be only partially attached to the garment shell 22, and can include a flap portion 80 unadhered to the garment shell 22 at a longitudinal end region 76 of the inner attachment member 70.

The garment shell 22 of the absorbent garment 10 of the present invention can, in various embodiments, include an inner attachment member 70 at either the front waist region 32 of the garment shell 22 or the back waist region 34 of the garment shell 22, or both. For example, the exemplary embodiments shown in FIGS. 2, 5-8 include a front inner attachment member 70 disposed at the front waist region 32 of the garment shell 22.

The inner attachment member 70 has a body-facing surface 72 and an opposite outward-facing surface 73. In particular embodiments, such as that illustrated in FIG. 6, the garment shell 22 is attached to the outward-facing surface 73 of the front inner attachment member 70, and the front fastening component 66 is attached to the body-facing surface 72 of the front inner attachment member 70. Alternatively, the configuration could be reversed (not shown).

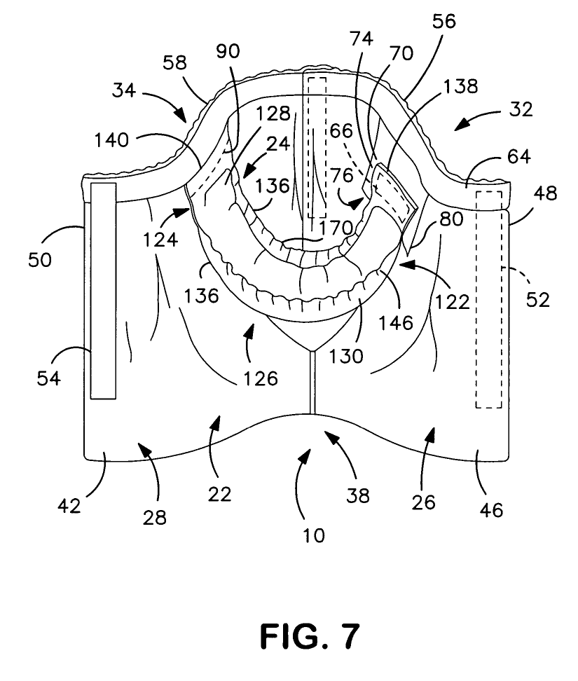

In those embodiments that include a front inner attachment member, the back waist end 140 of the absorbent assembly 24 can be attached to the back waist region 34 of the garment shell 22 in any number of ways. It can be permanently attached, as representatively illustrated in FIGS. 5 and 6. Alternatively, it can be removably attached, as representatively illustrated in FIG. 7. In that figure, the back waist region 124 of the absorbent assembly 24 includes a frangible line of weakness 90, such as perforations, to allow a user to disconnect at least a part of the absorbent assembly back waist region 124 from the absorbent garment 10. In yet another embodiment, as representatively illustrated in FIG. 8, the back waist end 140 of the absorbent assembly 24 can be refastenably attached to the garment shell 22, such as by engagement of fastening components 66 and 68. In still another embodiment, the back waist region 124 of the garment shell 22 can include a back inner attachment member 70, to which the back waist region 124 of the absorbent assembly 24 can be refastenably attached. In such an embodiment, the back inner attachment member 70 can, in certain configurations, itself include a frangible line of weakness (not shown) to allow a user to completely disconnect the absorbent assembly back waist region 124 from the absorbent garment 10. Of course, a front inner attachment member can likewise include a frangible line of weakness in certain embodiments.

The present invention may be better understood with reference to the following example.

### EXAMPLE

The following tests were conducted in order to demonstrate the types of materials that may be used to construct absorbent garments made in accordance with the present invention.

In this example, various garment shell materials and various outer cover materials for an absorbent assembly were tested against each other to determine a static and kinetic coefficient of friction. The garment shell materials were also tested against various standard bed sheet materials for static and kinetic coefficient of friction. Specifically, three different garment shell materials were tested against three types of outer covers for an absorbent assembly and three types of bed sheets.

The garment shell materials tested included the following:

1. Knit material: The knit material was obtained from knit boxers that was comprised of a knit fabric made from 100% cotton.

2. Spunbond web: The spunbond web had a basis weight of 1.5 osy and contained bicomponent filaments in a sheath-core arrangement. The sheath polymer comprised polyethylene while the core polymer comprised polypropylene. The polyethylene used was ASPUN® 6811A obtained from Dow Chemical. The polypropylene polymer used, on the other hand, was PP3155 obtained from the Exxon Corporation. The spunbond web was bonded with bond points that formed diamond-shaped patterns.

3. SMS Laminate: The third material tested was a spunbond/meltblown/spunbond laminate. The laminate had a basis weight of 1.0 osy. The spunbond webs used to form the above laminate were made from the same polymers as Sample 2 above. The spunbond webs in the laminate, however, had a basis weight of approximately 0.4 osy. The meltblown layer was also made from bicomponent filaments. The bicomponent filaments included a sheath polymer comprising DNDA-1082 NT-7 polyethylene obtained from Dow Chemical. The polypropylene core polymer, on the other hand, was PF-015 obtained from Basell. The laminate was drawn 30% and was bonded using a wire weave bond pattern. In a wire weave bond pattern, the bonding sites comprise small dash marks that are used in an alternating horizontal/vertical orientation.

The outer cover materials for an absorbent assembly that were tested included the following:

1. Polymer film: The polymer film tested was a polyethylene film. Specifically, the film had a thickness of 0.75 mil and was corona treated on both sides.

2. Stretch film laminate: The stretch film laminate included a film laminated to a spunbond web. In particular, the film was stretched for breathability and laminated to the spunbond web. The spunbond web had a basis weight of 0.475 osy. An adhesive in the amount of 1.0 gsm was applied in between the spunbond web and the film layer. The film layer had a basis weight of 0.5 osy. Stretch film laminates are described, for instance, in U.S. Pat. No. 6,177,607.

3. Polymer film/spunbond laminate: The polymer film/spunbond laminate included a spunbond web containing titanium dioxide particles. The spunbond web had a basis weight of 0.65 osy. The spunbond web was laminated to the polymer film described above. An adhesive was applied in between the two layers in a swirl pattern. The adhesive was DISPOMELT 34-5611 obtained from National Starch and was added at a rate of 0.05 grams per product.

The three standard bed sheet materials that were tested included the following:

1. Cotton/polyester bed sheet: The cotton/polyester bed sheet tested contained 50% cotton and 50% polyester. The bed sheet had a soft percale weave and had a 180 thread count.

2. Jersey sheet: The Jersey sheet tested was a 100% cotton Jersey knit bed sheet.

3. Flannel sheet: The flannel sheet tested was a 100% cotton heavyweight flannel sheet.

The materials were tested against each other for a static coefficient of friction and kinetic coefficient of friction according to the procedure described above. The following results were obtained.

| Interaction of Garment Shell | | STATIC |
|---|---|---|
| to Outer Cover | | COEFFICIENT OF |
| Garment Sheet | | FRICTION |
| Material | Outer Cover Material | Average |
| Knit material | Polymer Film | 0.517 |
| Knit material | Polymer Film/Spunbond | 0.6316 |
| | Laminate | |
| Knit material | SFL Laminate | 0.6618 |
| Spunbond web | Polymer Film | 0.377 |
| Spunbond web | Polymer Film/Spunbond | 0.3072 |
| | Laminate | |
| Spunbond web | SFL Laminate | 0.3054 |
| SMS laminate | Polymer Film | 0.4492 |
| SMS laminate | Polymer Film/Spunbond | 0.3978 |
| | Laminate | |
| SMS laminate | SFL Laminate | 0.426 |

| Interaction of Garment Shell | | Kinetic |
|---|---|---|
| to Outer Cover | | Coefficient of |
| Garment Shell | | Friction |
| Material | Outer Cover Material | Average |
| Knit material | Polymer Film | 0.48 |
| Knit material | Polymer Film/Spunbond | 0.5458 |
| | Laminate | |
| Knit material | SFL Laminate | 0.5566 |
| Spunbond web | Polymer Film | 0.3864 |
| Spunbond web | Polymer Film/Spunbond | 0.2704 |
| | Laminate | |
| Spunbond web | SFL Laminate | 0.2596 |
| SMS laminate | Polymer Film | 0.4038 |
| SMS laminate | Polymer Film/Spunbond | 0.3702 |
| | Laminate | |
| SMS laminate | SFL Laminate | 0.3652 |

| | | Static |
|---|---|---|
| Interaction of Garment Shell | | Coefficient of |
| to Bed Sheets | | Friction |
| Garment Shell Material | Sheets | Average |
| Knit material | Cotton/Poly | 0.8052 |
| Knit material | Flannel | 0.8598 |
| Knit material | Jersey | 0.8954 |
| Spunbond web | Cotton/Poly | 0.515 |
| Spunbond web | Flannel | 0.666 |
| Spunbond web | Jersey | 0.6286 |
| SMS laminate | Cotton/Poly | 0.6188 |
| SMS laminate | Flannel | 0.662 |
| SMS laminate | Jersey | 0.6938 |

| | | Kinetic |
|---|---|---|
| Interaction of Garment Shell | | Coefficient of |
| to Bed Sheets | | Friction |
| Garment Shell Material | Sheets | Average |
| Knit material | Cotton/Poly | 0.6326 |
| Knit material | Flannel | 0.73 |
| Knit material | Jersey | 0.785 |
| Spunbond web | Cotton/Poly | 0.4124 |
| Spunbond web | Flannel | 0.4602 |
| Spunbond web | Jersey | 0.5452 |
| SMS laminate | Cotton/Poly | 0.514 |
| SMS laminate | Flannel | 0.5324 |
| SMS laminate | Jersey | 0.6068 |

As shown above, the knit material generally had the highest coefficient of friction when tested against sheet materials. The spunbond web, on the other hand, had the lowest coefficient of friction, while the SMS laminate tended to fall inbetween the above two.

Although materials used in producing garments in accordance with the present invention should have coefficient of friction properties as described in the above specification, coefficient of friction between the various materials was found not to be the only attribute affecting the way a product looks. Drape, stiffness, the amount of static electricity that is built up in a material, and various other factors may also be taken into account when constructing the garment.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. An absorbent garment comprising:
a garment shell having a waist opening and at least one opposing leg opening for receiving the legs of a wearer, the garment shell including an interior and an exterior and having a first side facing towards the interior and a second side facing towards the exterior;
an absorbent assembly positioned on the interior of the garment shell, the absorbent assembly including an outer cover surrounding an absorbent member; and
wherein the outer cover of the absorbent assembly and the first side of the garment shell have a static coefficient of friction between them of from about 0.3 to about 0.7 and have a kinetic coefficient of friction between them of from about 0.2 to about 0.6.

2. An absorbent garment as defined in claim 1, wherein the garment shell includes two leg extensions that surround two opposing leg openings.

3. An absorbent garment as defined in claim 1, wherein the absorbent assembly further comprises an inner liner, the absorbent member being positioned in between the outer cover and the inner liner.

4. An absorbent garment as defined in claim 1, wherein the garment shell includes a waist area surrounding the waist opening, the absorbent assembly including a front end portion and a back end portion, the front end portion and the back end portion being connected to the waist area of the garment shell.

5. An absorbent garment as defined in claim 1, wherein the outer cover of the absorbent assembly comprises a spunbond web, a meltblown web, a film, or mixtures thereof.

6. An absorbent garment as defined in claim 1, wherein the garment shell comprises a spunbond web.

7. An absorbent garment as defined in claim 6, wherein the garment shell comprises a spunbond/meltblown/spunbond laminate or a spunbond/meltblown laminate.

8. An absorbent garment as defined in claim 7, wherein the garment shell comprises two layers of the laminate bonded together.

9. An absorbent garment as defined in claim 1, wherein the second side of the outer shell has a static coefficient of friction of from about 0.5 to about 0.9 and a kinetic coefficient of friction of from about 0.4 to about 0.8 when tested against a standard cotton/polyester sheet material.

10. An absorbent garment as defined in claim 1, wherein the outer cover of the absorbent assembly and the first side of the garment shell have a static coefficient of friction between them of from about 0.3 to about 0.4 and have a kinetic coefficient of friction between them of from about 0.2 to about 0.4.

11. An absorbent garment comprising:
a garment shell having a waist opening and at least one opposing leg opening for receiving the legs of a wearer, the garment shell including an interior and an exterior and having a first side facing towards the interior and a second side facing towards the exterior;
an absorbent assembly positioned on the interior of the garment shell, the absorbent assembly including an outer cover surrounding an absorbent member; and
wherein the static coefficient of friction between the first side of the garment shell and the outer cover of the absorbent assembly is at least about 15% less than the static coefficient of friction between the second side of the garment shell and a standard cotton/polyester sheet material and wherein the kinetic coefficient of friction between the first side of the garment shell and the outer cover of the absorbent assembly is also at least about 15% less than the kinetic coefficient of friction between the second side of the garment shell and a standard cotton/polyester sheet material.

12. An absorbent garment as defined in claim 11, wherein the static coefficient of friction between the first side of the garment shell and the outer cover of the absorbent assembly is at least about 20% less than the static coefficient of friction between the second side of the garment shell and a standard cotton/polyester sheet material and wherein the kinetic coefficient of friction between the first side of the garment shell and the outer cover of the absorbent assembly is at least about 20% less than the kinetic coefficient of friction between the second side of the garment shell and a standard cotton/polyester sheet material.

13. An absorbent garment as defined in claim 11, wherein the static coefficient of friction between the first side of the garment shell and the outer cover of the absorbent assembly is at least about 25% less than the static coefficient of friction between the second side of the garment shell and a standard cotton/polyester sheet material and wherein the kinetic coefficient of friction between the first side of the garment shell and the outer cover of the absorbent assembly is at least about 25% less than the kinetic coefficient of friction between the second side of the garment shell and a standard cotton/polyester sheet material.

14. An absorbent garment as defined in claim 11, wherein the static coefficient of friction between the first side of the garment shell and the outer cover is from about 0.3 to about 0.7 and the kinetic coefficient of friction between the first side of the garment shell and the outer cover is from about 0.2 to about 0.6 and wherein the static coefficient of friction between the second side of the garment shell and a standard cotton/polyester sheet material is from about 0.5 to about 0.9 and the kinetic coefficient of friction between the second side of the outer shell and a standard cotton/polyester sheet material is from about 0.4 to about 0.8.

15. An absorbent garment as defined in claim 11, wherein the static coefficient of friction between the first side of the garment shell and the outer cover is from about 0.3 to about 0.4 and the kinetic coefficient of friction between the first side of the garment shell and the outer cover is from about 0.2 to about 0.4 and wherein the static coefficient of friction between the second side of the garment shell and a standard cotton/polyester sheet material is from about 0.6 to about 0.7 and the kinetic coefficient of friction between the second side of the outer shell and a standard cotton/polyester sheet material is from about 0.4 to about 0.6.

16. An absorbent garment as defined in claim 11, wherein the garment shell includes two leg extensions that surround two opposing leg openings.

17. An absorbent garment as defined in claim 11, wherein the garment shell includes a waist area surrounding the waist opening, the absorbent assembly including a front end portion and a back end portion, the front end portion and the back end portion being connected to the waist area of the garment shell.

18. An absorbent garment as defined in claim 11, wherein the outer cover of the absorbent assembly comprises a spunbond web, a meltblown web, a film, or mixtures thereof.

19. An absorbent garment as defined in claim 11, wherein the garment shell comprises a spunbond web.

20. An absorbent garment as defined in claim 19, wherein the garment shell comprises a spunbond/meltblown/spunbond laminate or a spunbond/meltblown laminate.

21. An absorbent garment as defined in claim 20, wherein the garment shell comprises two layers of the laminate bonded together.

22. An absorbent garment as defined in claim 11, wherein the static coefficient of friction between the first side of the garment shell and the outer cover is less than about 0.35 and the kinetic coefficient of friction between the first side of the garment shell and the outer cover is less than about 0.3 and wherein the static coefficient of friction between the second side of the garment shell and a standard cotton/polyester sheet material is from about 0.6 to about 0.7 and the kinetic coefficient of friction between the second side of the outer shell and a standard cotton/polyester sheet material is from about 0.4 to about 0.6.

23. An absorbent garment comprising:
a garment shell having a waist opening and at least one opposing leg opening for receiving the legs of a wearer, the garment shell including an interior and an exterior and having a first side facing towards the interior and a second side facing towards the exterior, the garment shell comprising a laminate containing a spunbond web;
an absorbent assembly positioned on the interior of the garment shell, the absorbent assembly including a front end portion and a back end portion, the front end portion and the back end portion being connected to a waist area of the garment shell surrounding the waist opening, the absorbent assembly including an outer cover, an inner liner, and an absorbent member positioned in between the outer cover and the inner liner, the outer cover of the absorbent assembly comprising a spunbond web, a meltblown web, a film, or mixtures thereof; and
wherein the static coefficient of friction and the kinetic coefficient of friction between the first side of the garment shell and the outer cover of the absorbent assembly are less than the static coefficient of friction and the kinetic coefficient of friction between the second side of the garment shell and a standard cotton/polyester sheet material respectively, the static coefficient of friction between the first side of the garment shell and the outer cover of the absorbent assembly being from about 0.3 to about 0.7, the kinetic coefficient of friction between the first side of the garment shell and the outer cover of the absorbent assembly being from about 0.2 to about 0.6, the static coefficient of friction between the second side of the garment shell and a standard cotton/polyester sheet material being from about 0.5 to about 0.9 and the kinetic coefficient of friction between the second side of the outer shell and a standard cotton/polyester sheet material being from about 0.4 to about 0.8.

24. An absorbent garment as defined in claim 23, wherein the static coefficient of friction and the kinetic coefficient of friction between the first side of the garment shell and the outer cover of the absorbent assembly are both at least about 15% less than the static coefficient of friction and the kinetic coefficient of friction between the second side of the garment shell and a standard cotton/polyester sheet material.

25. An absorbent garment as defined in claim 23, wherein the static coefficient of friction and the kinetic coefficient of friction between the first side of the garment shell and the outer cover of the absorbent assembly are both at least about 20% less than the static coefficient of friction and the kinetic coefficient of friction between the second side of the garment shell and a standard cotton/polyester sheet material.

26. An absorbent garment as defined in claim 23, wherein the static coefficient of friction and the kinetic coefficient of friction between the first side of the garment shell and the outer cover of the absorbent assembly are both at least about 25% less than the static coefficient of friction and the kinetic coefficient of friction between the second side of the garment shell and a standard cotton/polyester sheet material.

27. An absorbent garment as defined in claim 23, wherein the garment shell includes two leg extensions that surround two opposing leg openings.

28. An absorbent garment as defined in claim 23, wherein the garment shell comprises a spunbond/meltblown/spunbond laminate or a spunbond/meltblown laminate.

29. An absorbent garment as defined in claim 23, wherein the garment shell comprises two layers of the laminate bonded together.

30. An absorbent garment as defined in claim 28, wherein the garment shell comprises two layers of the laminate bonded together.

31. An absorbent garment as defined in claim 23, wherein the static coefficient of friction between the first side of the garment shell and the outer cover is from about 0.3 to about 0.4 and the kinetic coefficient of friction between the first side of the garment shell and the outer cover is from about 0.2 to about 0.4 and wherein the static coefficient of friction between the second side of the garment shell and a standard cotton/polyester sheet material is from about 0.6 to about 0.7 and the kinetic coefficient of friction between the second side of the outer shell and a standard cotton/polyester sheet material is from about 0.4 to about 0.6.
